(19) United States
(12) Patent Application Publication
(10) Pub. No.: US 2005/0058611 A1
(43) Pub. Date: Mar. 17, 2005

(54) Preventing and/or combating collagen fiber degradation induced under conditions of natural exposure to sunlight

(75) Inventors: Dominique Fagot, Paris (FR); Francoise Bernerd, Paris (FR)

Correspondence Address: BURNS DOANE SWECKER & MATHIS L L P, POST OFFICE BOX 1404, ALEXANDRIA, VA, 22313-1404, US

(73) Assignee: L'OREAL, PARIS (FR)

(21) Appl. No.: 10/922,929

(22) Filed: Aug. 23, 2004

Related U.S. Application Data
(60) Provisional application No. 60/530,233, filed on Dec. 18, 2003.

(30) Foreign Application Priority Data
Aug. 22, 2003 (FR) 03/10103

(51) Int. Cl. C12Q 1/00; A61K 7/42

(52) U.S. Cl. 424/59; 435/4

(57) ABSTRACT

Cosmetic/dermatological compositions useful for preventing and/or combating the degradation of collagen fibers induced by solar radiation typically characterized by a UV-A/UV-B ratio ranging from 10 and 17, advantageously formulated for topical application onto the skin and/or scalp, contain a thus effective amount of at least one inhibitor of the production of photoinduced keratinocytic cytosoluble factors, notably sodium butyrate.

### CROSS-REFERENCE TO PRIORITY/PROVISIONAL APPLICATIONS

This application claims priority under 35 U.S.C. § 119 of FR 03/10103, filed Aug. 22, 2003, and of provisional application Ser. No. 60/530,233, filed Dec. 18, 2003, both hereby expressly incorporated by reference and both assigned to the assignee hereof. This application is also a continuation of said '233 provisional.

### BACKGROUND OF THE INVENTION

1. Technical Field of the Invention

The invention relates to the formulation, into a cosmetic or dermatological composition for preventing and/or combating the degradation of collagen fibers induced by solar radiation characterized by a UV-A/UV-B ratio of between 10 and 17, of an effective amount of at least one inhibitor of the production of photoinduced keratinocytic cytosoluble factors.

The invention also relates to the formulation, into a cosmetic or dermatological composition for preventing and/or combating the photoinduced degradation of collagen fibers, of inhibitors of specific keratinocytic cytosoluble factors.

Compositions comprising said inhibitors are suited to inhibit the production of MMP-1 in the dermis, induced in the presence of a suberythemal or erythemal natural sunlight exposure.

This invention also relates to the administration of compositions comprising particular inhibitors and to a cosmetic regime or regimen for preventing and/or reducing the signs of aging of the skin and/or intended to reduce hair loss.

According to the invention, the expression “natural sunlight exposure” especially means a sunlight exposure covering the real variations in solar radiation in terms of dose and UV-A/UV-B ratio, especially the zenithal condition, the said variations possibly corresponding to an “erythemal” sunlight exposure or to a “sub-erythemal” sunlight exposure, depending on whether or not it triggers an inflammatory erythema.

According to the invention, the expression “inhibitors of the “production” of keratinocytic cytosoluble factors” especially means any agent capable of inhibiting (i) the signaling pathways leading to the transcription or translation, (ii) the processes of post-translational modification, (iii) the secretion and/or (iv) the intracellular and/or extracellular activation of keratinocytic cytosoluble factors.

The same definition is applicable to collagenase “production” inhibitors.

2. Description of Background and/or Related and/or Prior Art

Human skin consists of two compartments, namely a surface compartment, the epidermis, and a deep compartment, the dermis.

Natural human epidermis is composed mainly of three types of cells: keratinocytes, which form the great majority, melanocytes and Langerhans cells. Each of these cell types contributes, by virtue of its intrinsic functions, toward the essential role played in the body by the skin.

The dermis gives the epidermis a solid support. It is also the epidermis' nourishing factor. It consists mainly of fibroblasts and of an extracellular matrix which is itself composed mainly of collagen, elastin and a substance known as ground substance, these components being synthesized by the fibroblast. Leukocytes, mastocytes and tissue macrophages are also found therein. It also contains blood vessels and nerve fibers.

These collagen fibers give the dermis its firmness. Collagen fibers consist of fibrils sealed together, thus forming more than 10 different types of structures. The firmness of the dermis is mainly due to the high level of entanglement of the collagen fibers packed together in all directions. The collagen fibers contribute toward the firmness, elasticity and tonicity of the skin and/or mucous membranes.

The collagen fibers are under constant renewal, but this renewal decreases with age, leading to thinning of the dermis.

In addition, various factors, including ultraviolet radiation, cause degradation of collagen, with all the consequences that may be envisaged on the structure and/or firmness of the skin and/or mucous membranes, in particular on the areas of the body usually exposed to sunlight: the face, the ears, the scalp, the neck, the forearms and the hands.

Damage to the skin associated with chronic exposure (repeated irradiation) or acute exposure (strong irradiation) to UV-A and/or UV-B has been well studied; in particular, it is known that:
UV-B rays (290-300 nm; 5% of the total UV rays), with the highest-energy wavelengths, especially affect the epidermal cells (keratinocytes), by acting on the DNA;
UV-A rays (320-400 nm; 95% of the total UV rays), which are more penetrating, reach the dermal cells such as the fibroblasts and act indirectly via the generation of free radicals;
furthermore, prolonged exposure to ultraviolet radiation, particularly to ultraviolet radiation of type A and/or B, has the effect of stimulating the expression of collagenases, particularly type 1 matrix metalloprotease (MMP-1).

This is one of the components of photoinduced aging of the skin. In addition, it is known that the activity of MMP-1, MMP-2 and MMP-9 increases with age and that this increase contributes, along with the slowing-down of cell growth, to the chronological aging of the skin (WO 98/36742).

Collagen fibers, although very strong, are, however, sensitive to certain enzymes.

These proteins form part of a family of enzymes known as matrix metalloproteases (MMPs), which are themselves members of a family of proteolytic enzymes (endoproteases) that have a zinc atom coordinated to 3 cysteine residues and one methionine residue in their active site and which can, collectively, degrade the macromolecular components of the extracellular matrix and of the basal layers at neutral pH (collagen, elastin, etc.).

The family of metalloproteases thus consists of several well-defined groups based on their resemblances in terms of structure and substrate specificity (see Woessner J. F., Faseb Journal, vol. 5, 1991, 2145). Among these groups, mention may be made of the collagenases intended to degrade fibrillar collagens (MMP-1 or interstitial collagenase, MMP-8 or neutrophilic collagenase, MMP-13 or collagenase 3), gelatinases that degrade type IV collagen or any form of denatured collagen (MMP-2 or gelatinase A (72 kDa), MMP-9 or gelatinase B (92 kDa), the stromelysins (MMP-3 or stromelysin 1, MMP-10 or stromelysin 2 and MMP-11 or stromelysin 3), whose broad spectrum of activity is directed towards the proteins of the extracellular matrix such as glycoproteins (fibronectin, laminin), proteoglycans, etc., matrilysin (MMP-7), metalloelastase (MMP-12) or membrane metalloproteases (MMP-14, MMP-15, MMP-16 and MMP-17).

The metalloproteases are produced and secreted in an inactive form (proenzyme). These “zymogenic” inactive forms are then activated in the extracellular environment by removing a propeptide region. The members of this family can activate each other. Regulation of the activity of the MMPs thus takes place at the level of expression of the genes (transcription and translation), at the level of activation of the zymogenic form, or at the level of local control of the active forms.

The importance of collagen in the structure of tissues, particularly the skin and/or mucous membranes, and the importance of preventing or combating its degradation in order thus to combat aging, whether this is chronobiological or photoinduced aging, and the consequences thereof, the thinning of the dermis and/or the development of flaccid and/or wrinkled skin, may thus be appreciated.

It is moreover known that MMPs are involved in the degradation of the perifollicular matrix and thus in hair loss.

It was hitherto considered that the process of photoaging of the skin and in particular the induction of metalloproteases responsible for the degradation of collagen fibers in the dermis, took place when the skin was subjected to UV radiation capable of inducing a solar erythema (inflammatory rash, sunburn), defined by a minimum erythemal dose (MED). This dose varies as a function of the individual's phototype and the UV-A/UV-B ratio.

Previous studies have thus sought to reproduce sunlight exposure conditions comprising UV-A and/or UV-B radiation, at doses around the MED, to identify compounds capable of protecting the skin against the harmful effects of sunlight, and in particular against erythema, indirectly associated with photoaging. However, to the present inventors' knowledge, none of the studies performed to date has made it possible to reproduce conditions close to natural exposure to sunlight. Specifically, in the studies of the prior art:
either the UV radiation is applied at “high” doses (for example 2MED, Laman et al., 2001, Photochem. Photobiol., 73, 657-663);
or the UV radiation is characterized by an imbalanced UV-A/UV-B ratio, very much in favor of the UV-B rays, which are responsible for erythema (100% UV-B, Fagot et al., 2002, Arch Dermatol Res., 293: 576-583).

There is thus a need to identify novel compounds that are effective, not only against erythema, but also under natural sunlight exposure conditions, i.e., against earlier and less perceptible events associated with the harmful effects of sunlight, for instance a gradual impairment in skin tissues, which is responsible, along with the time and repetition of the sunlight exposures, for the signs of photoaging of the skin (wrinkles, fine lines, flaccid, thinned skin, etc.). These early events according to the invention especially include degradation of the extracellular matrix and of proteins, for instance collagen and elastin, in particular degradation of collagen fibers induced by the action of metalloproteases and in particular MMP-1.

Lahman et al. have described, under conditions of simulated solar radiation SSR and at a dose equal to twice the minimum erythemal dose (2MED), which thus simulate conditions of erythemal sunlight exposure, a production of MMP-1 in the dermis (fibroblasts) and the epidermis (keratinocytes).

### SUMMARY OF THE INVENTION

It has now surprisingly and unexpectedly been determined that under conditions close to that of sub-erythemal natural sunlight exposure, i.e., under conditions of simulated solar radiation (SSR) at a sub-erythemal dose, and with a UV-A/UV-B ratio of between 10 and 17, keratinocytes are incapable of producing MMP-1, but nevertheless participate in the production of MMP-1 by the dermal fibroblasts, by means of a paracrine mechanism involving keratinocytic cytosoluble factors. In addition, it has now been shown that the stimulatory effect on the production of fibroblast MMP-1 is 18 times higher in the presence of photoinduced keratinocytic cytosoluble factors than in the case of direct irradiation of the fibroblasts (factor 8).

This is therefore the first time that the respective role of the epidermis and of the dermis in the production of MMP-1 in the dermis and the execution of a paracrine mechanism involving keratinocytic cytosoluble factors have been demonstrated, under conditions and at doses close to those of a natural exposure to sunlight, in particular under sub-erythemal conditions.

According to the invention, the expression “sub-erythemal dose” means a biologically effective dose (BED) capable of inducing the formation of sunburn cells in the epidermis, characterized especially by a round shape and a loss of connection with the keratinocytes, the said dose not inducing inflammatory erythema.

In particular, the BED dose according to the invention is 10 J/cm² in SSR, i.e., 9.2 J/cm² of UV-A and 0.8 J/cm² of UV-B, for an SSR of 2.34 mW/cm².

According to the invention, the term “simulated solar radiation” (SSR) means a simulated whole UV solar spectrum, comprising at least some UV rays of type A and of type B (290-400 nm).

This simulated solar radiation is generally obtained in the presence of a system combining at least one lamp and filters. A 1000 watt xenon lamp (Oriel corp. CT) equipped with Schoot UG 5.2 mm and WG 320/1.5 mm filters will be used, for example, to give a simulated solar UV spectrum (290-400 nm). Generally, the delivered spectral output corresponds to a real condition at the zenith (8% UV-B).

According to the invention, the term “paracrine mechanism” especially means a mechanism involving cytosoluble factors produced by epidermal cells (keratinocytes) and capable of diffusing as far as the target cells (fibroblasts) to prevent the production of MMP-1 by the said cells.

These results thus reflect new perspectives in the prevention and/or treatment of the early events associated with the harmful effects of sunlight, for instance the degradation of collagen fibers in the dermis, induced especially by natural solar radiation characterized by a UV-A/UV-B ratio of between 10 and 17. This UV-A/UV-B ratio covers variations in amounts of UV-A and UV-B radiation, depending on whether the prevailing conditions involve a large amount of UV-B (sun at the zenith, summer sunlight) or involve a small amount of UV-B and a large amount of UV-A (non-zenith condition).

It has also now been shown that the use of an effective amount of sodium butyrate makes it possible in vitro to reduce this stimulatory effect on MMP-1 production, by inhibiting the production of photoinduced keratinocytic cytosoluble factors.

The therapeutic use of sodium butyrate as an agent for inhibiting the proliferation of smooth muscles of the vascular system (U.S. Pat. No. 5,563,173) or as an anticancer agent (Saunders N et al., Cancer research, 59, 399-404, Jan. 15, 1999) is known in the prior art. EP-0,345,081 also describes oral tablets or ointments for treating chromatosis, containing calcium butyrate, another butyric acid salt, as an agent for inhibiting tyrosinase formation.

However, it has not at all been suggested to use histone deacetylase inhibitors and in particular butyric acid derivatives and/or salts for the preparation of cosmetic or dermatological compositions for preventing collagen degradation induced under sub-erythemal or erythemal sunlight exposure conditions.

The present invention thus features the formulation, into a cosmetic or dermatological composition for preventing and/or combating the degradation of collagen fibers induced by solar radiation characterized by a UV-A/UV-B ratio of between 10 and 17, of an effective amount of at least one inhibitor of the production of photoinduced keratinocytic cytosoluble factors.

The inhibitor of the production of photoinduced keratinocytic cytosoluble factors is formulated in the composition as an agent for preventing and/or combating at an early stage the degradation of collagen fibers induced under sub-erythemal or erythemal natural sunlight exposure conditions.

According to the invention, the term “solar radiation” especially means any radiation equivalent to the natural solar spectrum, in particular to the zenith spectrum, and comprising at least some UV-A and some UV-B in a UV-A/UV-B ratio of between 10 and 17, preferably greater than or equal to 11 and less than or equal to 16.

In particular, the solar radiation according to the invention may comprise 92% UV-A and 8% UV-B, reproducing the conditions of the zenith solar spectrum in the UV-B range. Preferably, the proportion of UV-B in the solar radiation defined according to the invention will be between 6% and 9%.

The treatment according to the invention thus makes it possible to effectively protect the skin throughout the day and thus to prevent and/or combat at an early stage the degradation of collagen fibers induced by sub-erythemal or erythemal natural sunlight exposure. Such treatment also has the advantage of overcoming the problems of bioavailability of conventional MMP inhibitors, due to the fact that the inhibitors of keratinocytic cytosoluble factors according to the invention have as their target epidermal cells (keratinocytes) rather than dermal cells.

The MMP inhibitors according to the invention will be termed “indirect” inhibitors, as opposed to the inhibitors known hitherto, which are termed “direct” inhibitors (action directly on the dermis, the site of production of MMP-1).

### DETAILED DESCRIPTION OF BEST MODE AND SPECIFIC/PREFERRED EMBODIMENTS OF THE INVENTION

The inhibition of the photoinduced degradation of collagen fibers in the dermis, under sub-erythemal or erythemal natural sunlight exposure conditions, especially makes it possible to envisage the following applications:
to prevent and/or reduce and/or treat the signs of photoaging of the skin, in particular thinning of the skin and/or loss of tonicity of the skin and/or loss of elasticity of the skin; and
indirectly to prevent and/or combat at an early stage menopause-related conditions, aggravated by the presence of photoinduced MMP-1;
to induce and/or stimulate hair growth and/or to reduce hair loss, aggravated by the presence of photoinduced MMP-1.

The expression “signs of aging of the skin” especially means any change in the outer appearance of the skin caused by chronobiological and/or photoinduced aging, for instance wrinkles and fine lines, wizened skin, flaccid skin, thinned skin, lack of elasticity and/or tonus of the skin, and also any internal change in the skin, for instance any internal degradation of the skin, in particular of collagen, following exposure to ultraviolet radiation.

The expression “menopause-related skin conditions” especially means thinned skin, a lack of elasticity and an accentuation of wrinkles and fine lines.

The reason for this is that, at the menopause, the main changes concerning the dermis are an impairment in the elastic tissue and a reduction in the collagen content and in the thickness of the dermis. In the case of menopausal women, this results in thinning of the skin and/or the mucous membranes. These women then have a sensation of “dry skin” or of taut skin, and an accentuation of the surface wrinkles and fine lines is observed. The skin has a coarse feel. Finally, the skin shows reduced suppleness.

The inhibitor of the production of photoinduced keratinocytic cytosoluble factors that is administered according to the invention may be selected according to the process comprising the following steps:
(a) normal human keratinocytes cultured as a monolayer are subjected to a simulated solar radiation or to a UV radiation, characterized by a UV-A/UV-B ratio of between 10 and 17 as described above;
(b) the irradiated or non-irradiated keratinocytes are placed in contact with a culture medium containing or not containing the test product;
(c) the conditioned medium obtained in (b) is placed in contact with normal human fibroblasts cultured as a monolayer;
(d) the production of interstitial collagenase MMP-1 in the fibroblast culture supernatant obtained in (c) is measured, and compared with a control consisting of a culture of normal human fibroblasts placed in contact with conditioned medium of non-irradiated keratinocytes; and
(e) the products for which the measurement of the production of interstitial collagenase MMP-1 is at least 10% less than that obtained for the control, preferably at least 20% less and even more preferably at least 30% less than that obtained for the control, are selected.

The term “conditioned medium” means a culture medium placed in contact with cells and from which the said cells are then taken. This medium thus contains the cytosoluble factors produced by the cells.

Other methods capable of demonstrating a difference in inhibition on the production of MMP-1 relative to a control may also be used, provided that the cellular models and the conditioned medium described are used. Examples that may be mentioned include the known methods for measuring the production of RNA (PCR, Northern blotting) or of proteins or of their enzymatic activities (Western blotting, ELISA, zymograms) applied to any epidermal cell model (reconstructed epiderms of EPISKIN type optionally containing Langherans cells, as described in EP-502,172 and EP-789,074) or dermal cells (fibroblasts in a monolayer or inserted into collagen lattices; or fibroblast mesenchymal stem cells).

The inhibitor of the production of photoinduced keratinocytic cytosoluble factors according to the invention is preferably selected from an inflammatory cytokine production inhibitor, a cyclooxygenase inhibitor and a histone deacetylase inhibitor, and mixtures thereof.

A histone deacetylase inhibitor will be used in particular.

The expression “inflammatory cytokine inhibitor” especially means any agent capable of inhibiting the production of interleukins IL1, IL6 and/or TNFalpha (Tumour Necrosis Factor); examples that may be mentioned include peroxysome proliferator-activated receptors (PPAR) activators, for instance clofibrate or WY14,643.

The term “cyclooxygenase inhibitor” is understood to denote any substance that makes it possible, in vivo, to limit or totally inhibit the transcription, translation and/or enzymatic activity of cyclooxygenases. The inhibitory activity on cyclooxygenases may be measured, for example, via HPLC chromatographic analysis methods (Huang M. et al., Cancer Res., 52, pp. 813-819,1991) or via radioimmunoassays (Lysz T. W. and Needleman P. J., Neurochim., 38, pp. 1111-1117, 1982). Mention may also be made of the test described in Salvameni et al., Proc. Natl. Sci. USA, 90, pp. 7240-7244, 1993).

Mention may be made, for example, of a cyclooxygenase-2 inhibitor, for instance Celecoxib. Other cyclooxygenase inhibitors may be selected from non-steroidal anti-inflammatory agents such as arylcarboxylic derivatives, pyrazole derivatives, oxicam derivatives and nicotinic acid derivatives.

Mention may also be made of meclofenamic acid, mefenamic acid, carprofen, diclofenac, diflunisal, fenbufen, fenoprofen, ibuprofen, indomethacin, ketoprofen, nabumetone, naproxen, sulindac, tenoxicam, tolmetin or acetylsalicylic acid.

Corticoids may also be used, such as dexamethasone or hydrocortisone, fenamates such as morniflumate, chalcone derivatives such as 3,4-dihydroxychalcone, or phospholipase A₂ inhibitors.

The term “histone deacetylase inhibitor” especially means α-lipoic acid, trichostatin A, and a C2 to C6 short-chain fatty acid such as propionic acid and/or its derivatives and/or its salts, butyric acid and/or its derivatives and/or its salts, and mixtures thereof.

According to one particular embodiment of the invention, any histone deacetylase inhibitor with the exception of α-lipoic acid will be used.

Among the “derivatives”, mention may be made especially of esters and in particular those comprising sugar and hydroxide radicals. Examples that may be mentioned include monoacetone glucose-3-propionate and monoacetone glucose-3-butyrate.

According to the invention, “salts” especially means organic or mineral salts. Among the organic salts that may be mentioned are ammonium salts obtained by reaction with basic amino acids.

Among the mineral salts that may especially be mentioned are the salts obtained by reaction with alkali metals (sodium or potassium salts), alkaline-earth metals (Mg²⁺, Ca²⁺) and transition metals (Fe, Mn, Co, Cu, etc.). Sodium propionate or sodium butyrate, and preferentially sodium butyrate, may be used, for example.

As butyric acid derivatives that may be used according to the invention, mention may be made of the compounds of general formula (I): 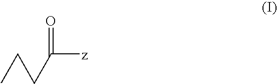
in which Z may be a group selected from —OH, —OR′, —NH2, —NHR′ and —NR′R″ groups; and R′ and R may be linear or branched, saturated or unsaturated C1-C8 alkyl or aryl groups, optionally substituted with hydroxyl, alkoxy, acyloxy, amino, alkylamino or aryl groups, and/or the salts thereof.

As organic salts of butyric acid or derivatives thereof of formula (I) that may be used according to the invention, mention may be made of triethanolamine, monoethanolamine, diethanolamine, hexadecylamine, N,N,N′,N′-tetrakis(2-hydroxypropyl)ethylenediamine and tris(hydroxymethyl)aminomethane salts, or alternatively carboxylic acid salts, for instance citrates, acetates, lactates, fumarates, gluconates and oxalates for the compounds according to the invention bearing a basic function.

As mineral salts of butyric acid or derivatives thereof of formula (I) that may be used according to the invention, mention may be made of the salts of mineral acids, for instance the sodium or potassium salts, the zinc (Zn²⁺), calcium (Ca²⁺), copper (Cu²⁺), iron (Fe²⁺, Fe³⁺), strontium (Sr²⁺), magnesium (Mg²⁺) and manganese (Mn²⁺) salts; or alternatively hydroxides, carbonates or hydrogen carbonates, chlorides, sulphates, phosphates or hydrogen phosphates for the compounds according to the invention bearing a basic function.

Sodium butyrate will preferably be used.

Among the keratinocytic cytosoluble factors induced in response to a UV irradiation as targets for the inhibitor that is the subject of the present invention, mention may be made especially of those described in Sesto A. et al., Proc. Natl. Sci. USA, 2002 Mar. 5; 99(5): 2965-70 and Li D. et al., FASEB J., 2001 November; 15(13): 2533-5.

This invention also features formulating, into a cosmetic or dermatological composition for preventing and/or combating the photoinduced degradation of collagen fibers, of an effective amount of at least one inhibitor of photoinduced keratinocytic cytosoluble factors, selected from a histone deacetylase inhibitor and a cyclooxygenase inhibitor, and mixtures thereof. The composition and/or the inhibitor will be useful as an inhibitor of metalloprotease (MMP) production, in particular as a collagenase MMP-1 inhibitor in the dermis. The term “inhibit the production of MMPs” means to reduce or even inhibit the production of MMPs.

Preferably, a histone deacetylase inhibitor selected from α-lipoic acid, trichostatin A, a C2 to C6 short-chain fatty acid such as propionic acid and/or its derivatives and/or its salts and butyric acid and/or its derivatives and/or its salts, and mixtures thereof, as defined above, will be used as inhibitor of keratinocytic cytosoluble factors.

According to one preferred embodiment, sodium butyrate or monoacetone 3-glucose butyrate will be used.

The inhibitor of photoinduced keratinocytic cytosoluble factors according to the invention will generally be used in an amount of between 10⁻¹²% and 5% of the total weight of the composition and preferably between 10⁻¹⁰% and 2% of the total weight of the composition. This amount corresponds to the amount that is effective to reduce or even inhibit collagen fiber degradation induced under sub-erythemal or erythemal natural sunlight conditions. This effective amount may be determined in vitro by measuring the reduction in MMP-1 production on cultures comprising keratinocytes and fibroblasts subjected to a simulated solar radiation or a UV radiation characterized by a UV-A/UV-B ratio of between 10 and 17.

The inhibitor of photoinduced keratinocytic cytosoluble factors according to the invention may be combined with another MMP inhibitor and also with standard cosmetic adjuvants, as defined below.

The use of the inhibitors of keratinocytic cytosoluble factors according to the invention is thus adapted to the preparation of compositions for reducing or even inhibiting the production of interstitial collagenase MMP-1 in the dermis, via a paracrine mechanism.

According to one particular embodiment of the invention, the inhibitor of the production of photoinduced keratinocytic cytosoluble factors as defined above is combined with at least one direct inhibitor of MMP-1 production.

These compositions will be useful to prevent and/or reduce the signs of aging of the skin and the degradation of collagen fibers of individuals subjected to sub-erythemal or erythemal sunlight exposure conditions.

The expression ““direct” inhibitor of production” means, as opposed to the inhibitor that is the subject of the invention and which is defined as being “indirect” inhibitor, an inhibitor that acts directly at the dermal level on the production and/or activation of MMP-1.

Examples that may be mentioned include inhibitors of MMP production of natural or synthetic origin.

The term “natural origin” means a metalloprotease inhibitor in pure form or as a solution at various concentrations, obtained via various extraction processes from a component of the animal and/or plant kingdom.

The term “synthetic origin” means the metalloprotease inhibitor in pure form or as a solution at various concentrations, obtained by chemical synthesis.

Examples of MMP inhibitors of natural origin that may be mentioned include:
a metalloprotease inhibitor selected from the MMP-1, MMP-2, MMP-3 and MMP-9 inhibitors;
a tissue inhibitor of metalloproteases (TIMP) such as the peptides known in the prior art under the names TIMP-1, TIMP-2, TIMP-3 and TIMP4 (Woessner J. F., Faseb Journal, 1991);
a natural extract containing ursolic acid or carotenoids or vitamin C or isoflavones, for instance genistein, which is known for its metalloprotease-inhibiting activity (U.S. Pat. No. 6,130,254);
lycopene or an isoflavone, and derivatives thereof. The activity of lycopene on metalloproteases has been described in EP-A-1,090,628; among the plant extracts containing it, mention may be made of soybean extracts (sold, for example, by Ichimaru Pharcos under the trade name Flavostérone SB®), red clover, flax, kakkon or sage;
a metalloprotease MMP-1 transcription inhibitor, such as retinol or its derivatives, and retinoic acid and its derivatives;
sapogenins, such as diosgenin, hecogenin, smilagenin, sarsapogenin, tigogenin, yamogenin and yuccagenin;
genistein and quercetin (U.S. Pat. Nos. 5,637,703, 5,665,367);
tetracyclines;
the malt extract sold by Coletica under the trade name Collalift®;
extracts of blueberry, rosemary or sage.

Examples of MMP inhibitors of synthetic origin that may be mentioned include:
adapalene or else similar peptides and/or Batimastat derivatives ((BB 94)=[4-(N-hydroxyamino)-2R-isobutyl-3S-(thiophen-2-ylthiomethyl)succinyl]-L-phenylalanine-N-methylamide), Marimastat ((BB 2516)=[2S-[N4(R*),2R*,3S]]-N4[2,2-dimethyl-1-[(methylamino)carbonyl]propyl]-N1,2-dihydroxy-3-(2-methylpropyl)butanediamide) sold by British Biotech;
tetracycline derivatives, such as minocycline, roliteracycline, chlortetracycline, methacycline, oxytetracycline, doxycycline, demeclocycline and the corresponding salts;
oligopeptides and lipopeptides, and lipoamino acids.

The uses described above are thus adapted to the preparation of compounds for inhibiting collagen fiber degradation induced in the presence of sub-erythemal or erythemal natural sunlight exposure.

The use of an inhibitor of photoinduced cytosoluble factors according to the invention will be advantageous as an agent for preventing the signs on the skin associated with photoinduced collagen degradation, this agent being effective, including when under sub-erythemal sunlight conditions. In particular, this use is intended to prevent thinning of the skin and/or loss of tonicity of the skin and/or loss of elasticity of the skin.

These compositions may especially be used on individuals with sensitive skin and/or a sensitive scalp, due to the efficacy of the inhibitors according to the invention under sub-erythemal sunlight exposure conditions.

Some people, termed as having sensitive skin or reactive skin, have in point of fact a very low level of cutaneous neurosensitivity and react to irritant substances or to environmental factors, including sunlight, much more strongly than other people; they complain of skin discomfort characterized by sensations of heating, stinging, tingling or non-allergic itching.

The present invention also features compositions comprising, in a physiologically acceptable medium, and according to a first alternative, an effective amount of at least one inhibitor selected from a cyclooxygenase inhibitor and a histone deacetylase inhibitor, and mixtures thereof.

The composition may be a cosmetic composition or a dermatological composition. It will preferably be a cosmetic composition.

According to the invention, the term “physiologically acceptable medium” means a medium that is compatible with the skin and/or its integuments (eyelashes, nails, hair) and/or mucous membranes, and in particular a medium that does not contain any solvent with an irritant effect.

In particular, the composition comprises an effective amount of at least one histone deacetylase inhibitor selected from trichostatin A, a C2 to C6 short-chain fatty acid such as propionic acid and/or its derivatives and/or its salts, and butyric acid and/or derivatives and/or its salts, and mixtures thereof.

Among the derivatives that may be mentioned are esters, and in particular those comprising sugar and hydroxide radicals, such as monoacetone glucose-3-propionate and monoacetone glucose-3-butyrate.

Among the salts that may be mentioned are organic salts, such as the ammonium salts obtained by reaction with basic amino acids, and the mineral salts obtained, for example, by reaction with alkali metals (sodium or potassium salts), alkaline-earth metals (Mg²⁺, Ca²⁺) and transition metals (Fe, Mn, Co, Cu, etc.).

The composition preferably comprises an effective amount of butyric acid and/or its derivatives and/or its salts, and in particular sodium butyrate. In particular, also, it is in the form of an aqueous-alcoholic or oily solution, an emulsion of oil-in-water or water-in-oil type or a multiple emulsion, an aqueous or anhydrous gel, microcapsules or microparticles, or vesicular dispersions of ionic and/or nonionic type.

As butyric acid derivatives that may be used in the compositions of the invention, mention may be made of the compounds of general formula (I): 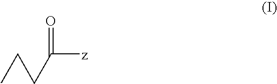
in which Z may be a group selected from —OH, —OR′, —NH2, —NHR′ and —NR′R″ groups; and R′ and R″ may be linear or branched, saturated or unsaturated C1-C8 alkyl or aryl groups, optionally substituted with hydroxyl, alkoxy, acyloxy, amino, alkylamino or aryl groups, and/or the salts thereof.

As organic salts of butyric acid or derivatives thereof of formula (I) that may be used in the compositions of the invention, mention may be made of triethanolamine, monoethanolamine, diethanolamine, hexadecylamine, N,N,N′,N′-tetrakis(2-hydroxypropyl)ethylenediamine and tris(hydroxymethyl)aminomethane salts, or alternatively carboxylic acid salts, for instance citrates, acetates, lactates, fumarates, gluconates and oxalates for the compounds according to the invention bearing a basic function.

As mineral salts of butyric acid or derivatives thereof of formula (I) that may be used in the compositions of the invention, mention may be made of the salts of mineral acids, for instance the sodium or potassium salts, the zinc (Zn²⁺), calcium (Ca²⁺), copper (Cu²⁺), iron (Fe²⁺, Fe³⁺), strontium (Sr²⁺), magnesium (Mg²⁺) and manganese (Mn²⁺) salts; or alternatively hydroxides, carbonates or hydrogen carbonates, chlorides, sulphates, phosphates or hydrogen phosphates for the compounds according to the invention bearing a basic function.

Preferably, the compositions of the invention will comprise any mineral salt of butyric acid with the exception of calcium butyrate. In particular, they will contain sodium butyrate.

The invention also relates to a cosmetic or dermatological composition comprising, in a physiologically acceptable medium, and according to a second alternative, at least an effective amount of an inhibitor of photoinduced keratinocytic cytosoluble factors as defined above, combined with at least one direct inhibitor of MMP-1 production that is active on the dermis.

In the context of this second alternative:
the inhibitor of photoinduced keratinocytic cytosoluble factors according to the invention is preferably selected from an inhibitor of inflammatory cytokine production, a cyclooxygenase inhibitor and a histone deacetylase inhibitor, such as α-lipoic acid, trichostatin A, propionic acid and/or its derivatives and/or its salts, in particular sodium propionate, and butyric acid and/or its derivatives and/or its salts, in particular sodium butyrate;
and the direct inhibitor of MMP-1 production that is active on the dermis may be selected from TIMP-1; lycopene or an isoflavone and derivatives thereof; retinol or its derivatives, retinoic acid and its derivatives; extracts of sage, blueberry or rosemary; adapalene or similar peptides and/or Batimastat derivatives ((BB 94)=[4-(N-hydroxyamino)-2R-isobutyl-3S-(thiophen-2-ylthiomethyl)succinyl]-L-phenyalanine-N-methylamide), Marimastat ((BB 2516)=[2S-[N4(R*),2R*,3S]]-N4[2,2-dimethyl-1-[(methylamino)carbonyl]propyl]-N 1,2-dihydroxy-3-(2-methylpropyl)butanediamide); sapogenins such as diosgenin, hecogenin, smilagenin, sarsapogenin, tigogenin, yamogenin and yuccagenin; genistein and quercetin; tetracycline and derivatives, such as minocycline, roliteracycline, chlortetracycline, methacycline, oxytetracycline, doxycycline, demeclocycline and corresponding salts; oligopeptides and lipopeptides, and lipoamino acids.

According to either of these alternatives, the said inhibitor of the production of photoinduced keratinocytic cytosoluble factors is generally in the composition in an amount of between 10⁻¹²% and 5% of the total weight of the composition and preferably between 10⁻¹⁰% and 2% of the total weight of the composition. This amount corresponds to the amount that is effective to reduce or even inhibit collagen fiber degradation induced under sub-erythemal or erythemal natural sunlight conditions. It may be determined in vitro as described above.

The composition may also advantageously comprise an agent selected from an agent that stimulates the synthesis of dermal macromolecules or that prevents their degradation, in particular an agent that stimulates collagen synthesis or prevents its degradation, an agent that promotes hair growth and/or limits hair loss, an antioxidant and a UV-screening agent, and mixtures thereof.

Among the “agents for stimulating dermal macromolecules or preventing their degradation”, mention may be made of those that act:
either on collagen synthesis, such as extracts of Centella asiatica; asiaticosides and derivatives; ascorbic acid or vitamin C and its derivatives; synthetic peptides such as iamin, biopeptide CL or the palmitoyloligopeptide sold by Sederma; peptides extracted from plants, such as the soybean hydrolysate sold by Coletica under the trade name Phytokine®; plant hormones such as auxins and lignans;
or on elastin synthesis, such as the extract of Saccharomyces cerivisiae sold by LSN under the trade name Cytovitin®; and the extract of the alga Macrocystis pyrifera sold by SECMA under the trade name Kelpadelie®;
or on glycosaminoglycan synthesis, such as the product of fermentation of milk with Lactobacillus vulgaris, sold by Brooks under the trade name Biomin yogourth®; the extract of the brown alga Padina pavonica sold by Alban Müller under the trade name HSP3®; and the extract of Saccharomyces cerevisiae available especially from the company Silab under the trade name Firmalift® or from the company LSN under the trade name Cytovitin®;
or on fibronectin synthesis, such as the extract of the zooplankton Salina sold by Seporga under the trade name GP4G®;
   the yeast extract available especially from the company Alban Müller under the trade name Drieline®; and the palmitoyl pentapeptide sold by Sederma under the trade name Matrixil®;
or on the inhibition of serine proteases such as leukocyte elastase or cathepsin G. Mention may be made of: the peptide extract of Leguminosa seeds (Pisum sativum) sold by LSN under the trade name Parelastyl®; heparinoids; and pseudodipeptides such as {2-[acetyl-(3-trifluoromethylphenyl)amino]-3-methylbutyrylamino}acetic acid.

As “agent for promoting hair growth and/or limiting hair loss”, mention may be made of aminexil, 6-0-[(9Z,12Z)octadeca-9,12-dienoyl]hexapyranose, benzalkonium chloride, benzethonium chloride, phenol, oestradiol, chlorpheniramine maleate, chlorophylline derivatives, cholesterol, cystein, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharidic or acyl-hexosaccharidic acids, aryl-substituted ethylenes, N-acyl amino acids, flavonoids, ascomycin derivatives and analogues, histamine antagonists, saponins, proteoglycanase inhibitors, oestrogen agonists and antagonists, pseudoterines, cytokines and growth factor promoters, IL-1 or IL-6 inhibitors, IL-10 promoters, TNF inhibitors, hydroxyl acids, benzophenones and hydantoin, and retinoic acid; vitamins such as vitamin D, vitamin B12 analogues and pantothenol; ceramides, in particular compounds of 2-aminoalkane-1,3-diol type and derivatives thereof, in particular 2-N-(2-hydroxyhexadecanoyl)aminooctadecane-1,3-diol and 2-oleoylaminooctadecane-1,3-diol; triterpenes, for instance ursolic acid and the compounds described in U.S. Pat. Nos. 5,529,769, 5,468,888 and 5,631,282; antipruriginous agents, for instance thenaldine, trimeprazine or cyproheptadine; antiparasitic agents, in particular metronidazole, crotamiton or pyrethroids; antifungal agents, in particular octopirox and compounds belonging to the imidazole class, such as econazole, ketoconazole or miconazole or salts thereof, nicotinic acid esters, especially including tocopheryl nicotinate, benzyl nicotinate and C₁-C₆ alkyl nicotinates, for instance methyl or hexyl nicotinate; calcium antagonists, for instance cinnarizine, diltiazem, nimodipine, verapamil, alverine and nifedipine; hormones such as oestriol or its analogues, thyroxine and its salts, and progesterone; antiandrogens such as oxendolone, spironolactone, diethylstilbestrol and flutamide; mixtures thereof.

Among the “antioxidants or free-radical scavengers” that may be mentioned, for example, are vitamin E and its derivatives such as tocopheryl acetate; bioflavonoids; coenzyme Q10 or ubiquinone; certain enzymes, for instance catalase, superoxide dismutase, lactoperoxidase, glutathione peroxidase and quinone reductases; glutathione; benzylidene camphor; benzylcyclanones; substituted naphthalenones; pidolates; phytanetriol; gamma-oryzanol; lignans; and melatonin.

The concentration of these additional agents in the compositions according to the invention may range from 0.0001% to 20% by weight and is preferably between 0.001% and 5% by weight relative to the total weight of the composition.

The term “UV-screening agents” means UV-A-active and/or UV-B-active mineral photoprotective agents (absorbers), which are water-soluble or liposoluble, or else insoluble in the cosmetic solvents commonly used, or organic photoprotective agents.

The organic photoprotective agents are selected especially from anthranilates; cinnamic derivatives; dibenzoylmethane derivatives; salicylic derivatives; camphor derivatives; triazine derivatives such as those described in U.S. Pat. No. 4,367,390, EP-863,145, EP-517,104, EP-570,838, EP-796,851, EP-775,698, EP-878,469, EP-933,376, EP-507,691, EP-507,692, EP-790,243, EP-944,624; benzophenone derivatives; β,β-diphenylacrylate derivatives; benzotriazole derivatives; benzalmalonate derivatives; benzimidazole derivatives; imidazolines; bis-benzazolyl derivatives as described in EP-669,323 and U.S. Pat. No. 2,463,264; p-aminobenzoic acid (PABA) derivatives; methylenebis(hydroxyphenylbenzotriazole) derivatives as described in U.S. Pat. Nos. 5,237,071, 5,166,355, GB-2,303,549, DE-197,26,184 and EP-893,119; screening polymers and screening silicones such as those described especially in patent application WO 93/04665; dimers derived from α-alkylstyrene, such as those described in DE-198,55,649; 4,4-diarylbutadienes such as those described in EP-0,967,200, DE-197,46,654, DE-197,55,649, EP-A-1,008,586, EP-1-133,980 and EP-133,981, and mixtures thereof.

The organic photoprotective agents that are more particularly preferred are selected from the following compounds: ethylhexyl salicylate, ethylhexyl methoxycinnamate, octocrylene, phenylbenzimidazole sulphonic acid, benzophenone-3, benzophenone-4, benzophenone-5, 4-methylbenzylidene camphor, terephthalylidene dicamphor sulphonic acid, disodium phenyl dibenzimidazole tetrasulphonate, 2,4,6-tris(diisobutyl 4′-aminobenzalmalonate)-s-triazine, anisotriazine, ethylhexyl triazone, diethylhexylbutamido triazone, methylenebisbenzotriazolyl tetramethylbutylphenol, drometrizole trisiloxane, 1,1-dicarboxy(2,2′-dimethylpropyl)4,4-diphenylbutadiene, and mixtures thereof.

The mineral photoprotective agents are selected from pigments or nanopigments (mean size of the primary particles: generally between 5 nm and 100 nm and preferably between 10 nm and 50 nm) of coated or uncoated metal oxides such as, for example, nanopigments of titanium oxide (amorphous or crystallized in rutile and/or anatase form), of iron oxide, of zinc oxide, of zirconium oxide or of cerium oxide, which are all UV photoprotective agents that are well known per se. Standard coating agents are, moreover, alumina and/or aluminum stearate. Such coated or uncoated metal oxide nanopigments are described in particular in EP-518,772 and EP-518,773.

The photoprotective agents are generally present in the compositions according to the invention in proportions ranging from 0.1% to 20% by weight relative to the total weight of the composition, and preferably ranging from 0.2% to 15% by weight relative to the total weight of the composition.

Ideally, the inhibitor of the production of keratinocytic cytosoluble factors according to the invention and the other agents may be combined in cosmetic care preparations for areas exposed to sunlight (scalp, body, face, lips) and, in general, in any cosmetic preparations for combating aging of the skin and for combating hair loss, whose aim is to slow down the chronobiological destructuring of the support tissues and the architecture of the cutaneous and capillary matrix components.

A preferred composition of the invention is a composition for cosmetic use.

This composition may be in any known galenical form adapted to the mode of use, in particular adapted to topical application to the skin and/or the scalp.

According to one particular embodiment of the invention, the composition according to the invention is intended for oral administration, in particular as an “oral cosmetic”: it may especially be in the form of wafer capsules, gel capsules, sugar-coated tablets, granules, chewing pastes, gels, drinkable syrups, tablets or any other form known to those skilled in the art. In particular, the active agent(s) according to the invention may be incorporated into any form of food supplement or enriched food, for example dietary bars, or compacted or non-compacted powders. The powders may be dilutable in water, in soda, dairy products or soybean derivatives, or may be incorporated into dietary bars.

The active agents may be formulated with the excipients and components that are usual for such oral compositions or food supplements, i.e., especially, fatty and/or aqueous components, humectants, thickeners, preservatives, texturing agents, flavorings and/or coatings, antioxidants, preservatives and dyes that are common in the food sector.

The formulating agents and excipients for an oral composition, and especially for food supplements, are known in this field and are not described in detail herein.

According to another particular embodiment of the invention, the composition according to the invention is intended for topical application to the skin: it may be in the form of an aqueous, alcoholic or aqueous-alcoholic solution or suspension, or an oily suspension, a dispersion, an emulsion obtained by dispersing a fatty phase in an aqueous phase (O/W) or conversely (W/O) or a multiple emulsion, an aqueous, aqueous-alcoholic or oily gel, microcapsules or microparticles, or vesicular dispersions of ionic and/or nonionic type.

In particular, the composition for topical application may be in the form of an aqueous, aqueous-alcoholic or oily solution or in the form of a dispersion of the lotion or serum type, emulsions of liquid or semi-liquid consistency of the milk type, obtained by dispersing a fatty phase in an aqueous phase (O/W) or conversely (W/O) or multiple emulsions, a free or compacted powder to be used in unmodified form or to be incorporated into a physiologically acceptable medium, or suspensions or emulsions of soft consistency of the aqueous or anhydrous cream or gel type, or alternatively microcapsules or microparticles, or vesicular dispersions of ionic and/or nonionic type. It may thus be in the form of a cream, a powder, an impregnated pad, a solution, an emulsion, a vesicular dispersion, a lotion, a gel, a spray, a suspension, a shampoo, an aerosol or a mousse. It may also consist of solid preparations constituting soaps or cleansing bars.

These compositions are prepared according to the usual methods.

Preferably, it will be a day composition or an antisun composition with a protective effect against collagen fiber degradation induced by exposure to sunlight, even a sub-erythemal exposure.

The composition according to the invention may also be intended for topical application to the scalp; it may be in the form of a hair care lotion, a gel, a shampoo or a hair conditioner, a cleansing liquid or solid soap, a product for shaping the hairstyle (lacquer, hair setting product or styling gel), a medicated mask, or a composition for permanent-waving, relaxing, dyeing or bleaching the hair.

The amounts of the various constituents of the physiological medium of the composition of the invention are those generally used in the fields under consideration. In addition, these compositions are prepared according to the usual methods.

When the composition is an emulsion, the proportion of the fatty phase may range from 2% to 80% by weight and preferably from 5% to 50% by weight relative to the total weight of the composition.

The fatty phase may contain fatty or oily compounds that are liquid at room temperature (25° C.) and atmospheric pressure (760 mmHg), which are generally known as oils. These oils may or may not be mutually compatible and may form a macroscopically homogeneous liquid fatty phase or a two-phase or three-phase system. In addition to the oils, the fatty phase may contain waxes, gums, lipophilic polymers, “pasty” or viscous products containing solid parts and liquid parts.

The aqueous phase contains water and optionally an ingredient that is miscible in all proportions with water, for instance C₁ to C₈ lower alcohols such as ethanol or isopropanol, polyols, for instance propylene glycol, glycerol or sorbitol, or alternatively acetone or ether.

The emulsifiers and co-emulsifiers used to obtain a composition in emulsion form are those generally used in the cosmetic and pharmaceutical fields. Their nature also depends on the sense of the emulsion. In practice, the emulsifier and the optional co-emulsifier are present in the composition in a proportion ranging from 0.1% to 30% by weight, preferably from 0.5% to 20% by weight and better still from 1% to 8% by weight. The emulsion may also contain lipid vesicles and especially liposomes.

As emulsifiers that may be used in the invention, examples that may be mentioned include fatty acid esters of polyethylene glycol, such as PEG-100 stearate, and fatty acid esters of glycerol, such as glyceryl stearate.

As oils that may be used in the invention, mention may be made of oils of mineral origin (liquid petroleum jelly or hydrogenated isoparaffin), oils of plant origin (liquid fraction of shea butter, sunflower oil, apricot oil, fatty alcohol or fatty acid), oils of animal origin (perhydrosqualene), synthetic oils (fatty acid ester, or purcellin oil), silicone oils (linear or cyclic polydimethylsiloxane, or phenyl trimethicone) and fluoro oils (perfluoropolyethers). Waxes that may be mentioned include silicone waxes, beeswax, carnauba wax or paraffin wax and polyethylene wax.

In known manner, the composition used according to the invention may also contain adjuvants that are common in the fields under consideration, such as preservatives, antioxidants, solvents, aqueous-phase or oily-phase thickeners or gelling agents, fragrances, fillers, pigments, odor absorbers, dyestuffs, electrolytes, neutralizers and cosmetic and pharmaceutical active agents, and mixtures thereof.

The amounts of these various adjuvants are those conventionally used in the field under consideration, for example from 0.01% to 50%, better still from 0.1% to 20% and for example from 0.1% to 10% of the total weight of the composition. Depending on their nature, these adjuvants may be introduced into the fatty phase, into the aqueous phase or into lipid vesicles.

Needless to say, one skilled in the art will take care to select the optional additional additives and/or the amount thereof such that the advantageous properties of the composition according to the invention, i.e., the inhibition of collagenase expression in the dermis, are not, or not substantially, adversely affected by the envisaged addition.

The invention also relates to a cosmetic regime or regimen for preventing and/or reducing and/or treating the signs of photo-aging of the skin and/or menopause-related conditions, comprising the oral administration or the topical application to the skin of a cosmetic composition as defined above.

In particular, the process is directed towards preventing and/or treating any change in the outer appearance of the skin caused by photoinduced aging, for instance wrinkles and fine lines, wizened skin, flaccid skin, thinned skin, and lack of elasticity and/or of tonus of the skin.

This invention also features a cosmetic regime or regimen for inducing and/or stimulating hair growth and/or for reducing hair loss, comprising orally administering or topically applying to the scalp a cosmetic composition as defined above, leaving the said composition in contact with the scalp, and optionally rinsing the scalp.

Preferably, the application will be performed daily in the morning (before any exposure to natural solar radiation), especially to the parts of the body usually exposed to sunlight, for instance the face, the ears, the neck, the forearms, the hands and the scalp.

These applications may be repeated over one or more months depending on the individual.

The invention will be described in greater detail by the non-limiting examples below.

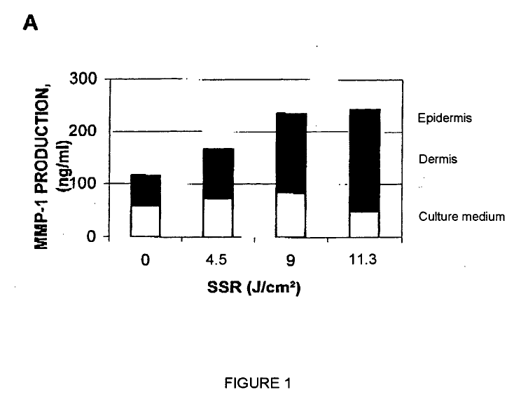

FIG. 1 shows the effect of an SSR exposure for 24 hours on the production of MMP-1 of a reconstructed human skin in vitro, by ELISA measurement.

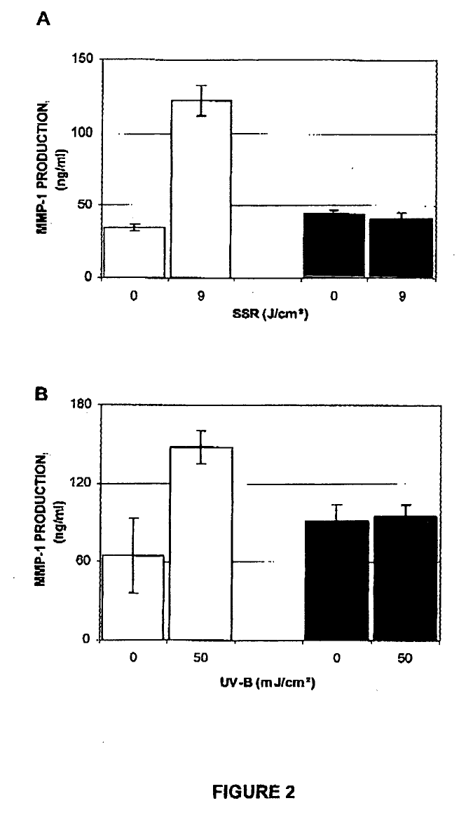

FIG. 2 illustrates the role of the epidermis in the production of MMP-1 of a reconstructed skin in vitro. The reconstructed human skins were exposed to SSR or UV-B and the production of MMP-1 is evaluated by ELISA 24 hours later. Alternatively, the epidermis of the reconstructed skin was removed immediately after UV exposure (black bars).

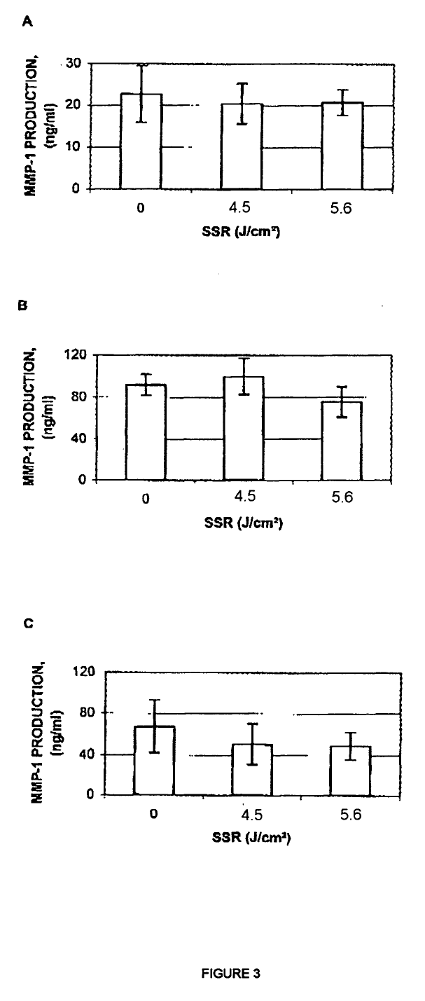

FIG. 3 shows the effects of an SSR exposure on the production of MMP-1 in an Episkin™ reconstructed epidermis. The MMP-1 content is evaluated by ELISA 24 hours later in the epidermis (A), in the acellular collagen gel (B) and in the culture medium (C).

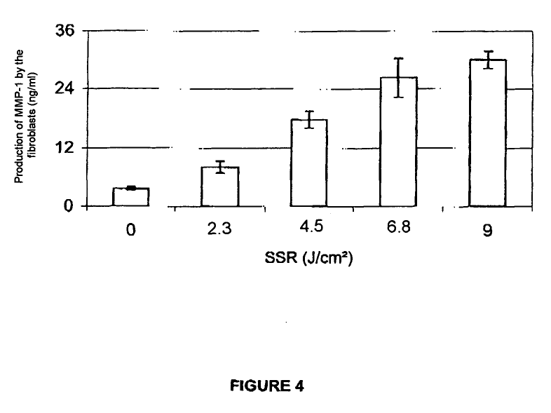

FIG. 4 is a histogram showing the effect of an SSR exposure on the production of MMP-1 in normal human dermal fibroblasts.

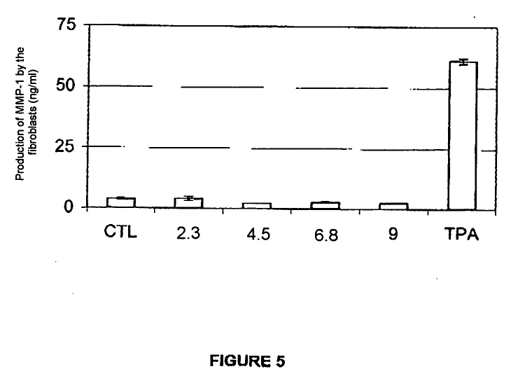

FIG. 5 illustrates the production of MMP-1 by normal human epidermal keratinocytes. The cells cultured on plastic are exposed to SSR or treated with TPA (20 ng/ml). The MMP-1 content is evaluated by ELISA 24 hours after treatment in the cell culture supernatant.

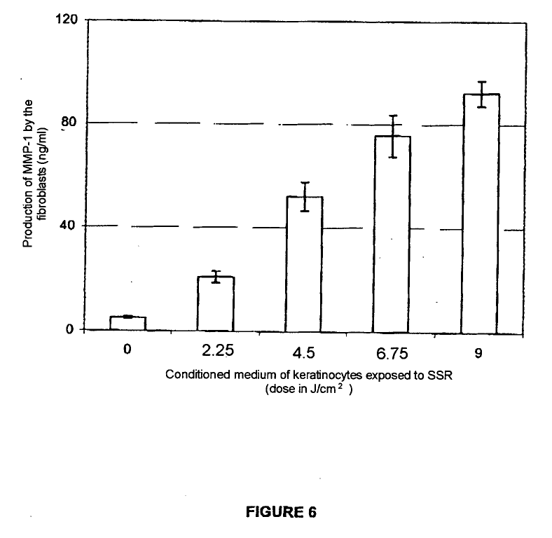

FIG. 6 is a histogram representing the effect of the conditioned medium of keratinocytes exposed to SSR on the production of MMP-1 in normal human dermal fibroblasts. The conditioned medium is collected 24 hours after irradiation and transferred onto normal human dermal fibroblasts. The production of MMP-1 is evaluated in the culture medium of the dermal fibroblasts by ELISA, 24 hours after transfer.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Example 1

### Demonstration of the Role of Keratinocytic Cytosoluble Factors in the Production of MMP-1 by the Fibroblasts, Under Conditions of Simulated Natural Sunlight Exposure (SSR)

Materials and Methods:

Cell Cultures:

Human keratinocytes are isolated from normal human skin (breast surgery) and cultured on a nourishing layer of mitomycin-treated 3T3 mouse fibroblasts.

The human dermal fibroblasts are isolated from amplified mammary skin explants and cultured on Dulbecco's Modified Eagle's Medium (DMEM) containing 10% FCS, supplemented with glutamine (2 mM), sodium pyruvate (1 mM) and non-essential amino acids.

All the cell cultures are maintained at 37° C. under 5% CO₂.

The reconstructed skins are composed of one equivalent of live dermis and of a differentiated epidermis, prepared as described in Asselineau et al. (1985), Exp. Cell. Res., 159, 536-539.

A model of reconstructed human epidermis, Episkin™ (Episkin SNC, France), was also used. Human mammary keratinocytes were inoculated onto a cell-free collagen substrate and grown to complete epidermal differentiation.

An “ex vivo” human skin is obtained from breast surgery. 6 mm biopsies of human skin were prepared and maintained for 24 hours in culture at the air-liquid interface on a grille (ex vivo) under the same conditions as for a reconstructed skin.

Simulated Solar Radiation (SSR):

A simulated solar radiation (SSR) is obtained using a 1000 watt xenon lamp (Oriel corp. CT) equipped with Schoot UG 5/2 mm and WG 320/1.5 mm filters. This filtered xenon source provides a simulated solar UV spectrum (290-400 nm). The delivered spectral output is 9% of UV-B, corresponding to a real solar condition at the zenith. A Philips TL 20 W/12 tube equipped with a Kodacel filter to remove the wavelengths below 290 nm is used as UV-B source. The wavelength spectrum is checked scrupulously with a Macam SR3010 spectroradiometer.

Just before irradiation, the culture medium of the sub-confluent cells (80-90% of confluence) is replaced with a PBS phosphate buffer to avoid the generation of phototoxic products by the medium. Under these conditions, no loss of viability of the cells is observed at the UV doses used.

After the irradiation, the PBS buffer is replaced with the keratinocyte medium or the fibroblast medium free of serum or of growth factors known to interfere with the detection or production of MMP-1.

Biochemical Analysis of MMP-1 Production:

ELISA (Detection):

The MMP-1 content is measured by ELISA (Biotrak kit RPN 2610, Amersham Biotech, France) according to the supplier's instructions.

Western Blotting (Quantitative Analysis):

The production of MMP-1 is also evaluated by the standard Western blotting method under reductive conditions on 10% polyacrylamide gel, transferred onto Hybond N membranes (Amersham Biotech).

The membranes are incubated with an anti-human MMP-1 mouse monoclonal antibody for 1 hour at room temperature and then washed.

The membranes are then incubated with a peroxidase-bound anti-mouse antibody for 45 minutes at room temperature, washed and stained in the presence of a chemoluminescent system ECL (Amersham Biotech).

Zymograms (Enzymatic Activity):

Novex zymogram gels were used to study the metalloproteases via detection of their proteolytic activity.

The samples were analysed by SDS-PAGE without heating or reduction, containing casein blue to detect collagenase. After gel electrophoresis, the gels are washed twice in a renaturing buffer for zymograms (Biorad). The caseinolytic proteins are identified by light bands on a dark background after labeling the proteins with coomassie blue (Pierce, France).

Results:

Determination of a Biologically Effective Dose on Skin Ex-Vivo:

In human skin in vivo, an exposure to sunlight induces the formation in the epidermis of “sunburn” cells, identified by a round shape, loss of connection with the keratinocytes, a suprabasal localization, and an eosinophilic cytoplasm around a dense and contracted nucleus.

On a human skin ex vivo, histological analyses of samples taken 24 hours after exposure to a simulated solar radiation reveal the appearance of “sunburn” cells in the suprabasal layer of the epidermis. The dose-response experiments determined a biologically effective dose (BED) of about 10 J/cm²: this dose corresponds to the minimum SSR dose inducing the formation of “sunburn” cells.

The production of MMP-1 in the culture medium after UV irradiation was thus evaluated using doses of simulated solar radiation (SSR) at about the biologically effective dose (BED). The ELISA and Western blotting results show that the SSR exposure induces an increase in the level of secreted MMP-1 protein, in a dose-dependent manner. Analysis of the zymogram reveals that this effect is associated with an increase in the proteolytic activity of MMP-1 in the culture medium.

Production of MMP-1 using a Reconstructed Skin In Vitro Subjected to SSR:

As for the ex vivo skin, the formation of “sunburn” cells is detected on a reconstructed skin in vitro at an SSR dose of 9 J/cm² and is also dose-dependent.

In response to an exposure of SSR type, a dose-dependent increase in the content of MMP-1 protein may be detected by ELISA in the culture medium and the equivalent dermis, but not in the epidermis (FIG. 1).

The Western blots confirm this result; furthermore, the Western blot analysis also reveals that the MMP-1 protein is found in its inactive form proMMP (zymogen) and its active form in the dermis equivalent, but mainly in its inactive form in the culture medium.

Analysis of the zymogram confirms an increase in the proteolytic activity after SSR exposure in the dermis and to a lesser extent in the culture medium, but not in the epidermis.

These results indicate that the localization of MMP-1 induced by SSR exposure is preferably in the dermis.

It is moreover known that:

UV-A radiation alone is capable of inducing the production of MMP-1 in a reconstructed human skin in vitro, via a direct effect of the UV-A on the dermal fibroblasts (Bernerd F et al. 1998, Cell Death Differ, 5, 792-802);
UV-B radiation, whose target is preferably the epidermis due to its poorer penetration, used on a reconstructed skin in vitro, at a biologically effective dose, is also capable of inducing a production of MMP-1 detected in the culture medium of irradiated samples.

However, the removal of the epidermal layer by peeling, just after the exposure of SSR or UV-B source type cancels the production of MMP-1, suggesting an essential participation of the epidermis in this induction (FIG. 2).

The following experiments were performed to determine the direct and/or indirect role of the epidermis in this induction.

Production of MMP-1 using a Reconstructed Epidermis In Vitro Subjected to SSR:

An exposure of SSR type of the Episkin™ model to a dose capable of inducing the formation of “sunburn” cells does not lead to induction of the production of MMP-1 in the epidermal leaflet, nor in the acellular collagen gel nor in the Episkin™ culture medium (FIG. 3).

Taken together with the preceding results, these results suggest that the dermal fibroblasts play an essential role in the production of MMP-1 after an exposure of SSR type and that the epidermis, although not responsible for a direct production of MMP-1, would appear to participate indirectly in the induction of MMP-1 after an exposure of SSR type.

Production of MMP-1 using Separate Keratinocyte and Fibroblast Cultures, Subjected to SSR:

Previous studies have documented in depth the capacity of UV-A and UV-B used separately to stimulate the production of MMP-1 in fibroblast cultures.

In agreement with these studies, it was possible to show that 24 hours after an exposure of SSR type, the normal human dermal fibroblasts growing on plastic showed a dose-dependent increase in MMP-1 production in the cell culture supernatant, quantified by ELISA or detected by Western blotting and zymographic analysis (FIG. 4).

To determine whether the irradiation of SSR type could also modulate the production of MMP-1 by keratinocytes growing on plastic, the cells were exposed to an SSR radiation. The MMP-1 extracellular contents were quantified 24 hours after UV radiation. In contrast with the fibroblasts, the exposure of SSR type on the keratinocytes does not lead to an increase in MMP-1 production. However, stimulation of the keratinocytes with TPA (Trans Plasminogen activator, a protein kinase C activator), used as positive control, leads to an accumulation of MMP-1 production (FIG. 5).

These results indicate that the keratinocytes are capable of producing MMP-1 in response to an appropriate treatment, but not in response to an SSR exposure.

Action of the Epidermis (Keratinocytes) on the Production of MMP-1 by Dermal Fibroblasts Via a Paracrine Mechanism:

To test the involvement of a paracrine mechanism, given that the production of MMP-1 by fibroblasts is regulated by positive modulators such as cytokines, normal human keratinocytes were exposed to a radiation of SSR type and the culture media were collected 24 hours later.

Next, the human dermal fibroblasts are treated with the conditioned medium obtained from the keratinocytes exposed to SSR; this conditioned medium contains keratinocytic cytosoluble factors, and especially photoinduced cytosoluble factors.

The MMP-1 content is then evaluated in the culture medium of the fibroblasts 24 hours later.

The results show that the conditioned medium of the keratinocytes exposed to SSR is capable of positively regulating the production of MMP-1 by the human fibroblasts in a dose-dependent manner, detected by ELISA (FIG. 6).

Under the experimental conditions, the production of MMP-1 by the dermal fibroblasts, induced by the conditioned medium, is markedly greater than the production of MMP-1 directly induced by the radiation of SSR type (18-fold as opposed to 8-fold).

These data indicate that the development of cell damage in the dermis, induced by a solar radiation, would appear to be due to specific direct effects in the dermis, mainly induced by penetrating UV-A wavelengths. However, a significant participation of the epidermal compartment is involved in releasing keratinocytic soluble factors, which is thought to be greatly influenced by the less penetrating UV-B wavelengths.

### Example 2

### Selection of Inhibitors of the Production of Photoinduced Keratinocytic Cytosoluble Factors

Normal human keratinocytes cultured as a monolayer on plastic were subjected to a UV-B radiation (10 mJ/cm²). The experiment may also be performed in the presence of an SSR as defined in Example 1.

A Philips TL 20 W/12 tube equipped with a Kodacel filter to remove the wavelengths below 290 nm is used as UV-B source.

Just before irradiation, the culture medium of the sub-confluent cells (80-90% of confluence) is replaced with a PBS phosphate buffer to avoid the generation of phototoxic products by the medium. Under these conditions, no loss of viability of the cells is observed at the UV doses used.

After irradiation, the cells were placed in contact with an MEM culture medium free of serum and of growth factor, which are known to interfere with the detection or production of MMP-1, containing or not containing sodium butyrate NaBu (3 mM).

The conditioned medium of the keratinocytes (irradiated or not, in the presence or absence of sodium butyrate) was taken up 24 hours after irradiation and placed in contact with normal human fibroblasts.

The production of MMP-1 detected 24 hours later in the fibroblast culture supernatant was quantified by means of the “Biotrak MMP-1 human ELISA system” detection kit sold by Amersham, according to the supplier's indications.

The results are read using a LabSystems microplate reader equipped with a 450 nm filter.

The amount of MMP-1 is calculated relative to a calibration range prepared with a standard supplied with the kit, and is expressed in ng/ml.

The results are as follows:

| | Production of |
|---|---|
| Experimental conditions | MMP-1 (ng/ml) |
| Non-irradiated keratinocyte control conditioned | 13.80 ± 1.01 |
| medium | |
| Non-irradiated keratinocyte conditioned medium, | 4.84 ± 0.36 |
| treated with sodium butyrate (NaBu) | |
| Irradiated keratinocyte conditioned medium | 40.70 ± 0.64 |
| Irradiated keratinocyte conditioned medium, treated | 16.97 ± 3.7 |
| with sodium butyrate (NaBu) | |

In the control condition (non-irradiated), the production of MMP-1 corresponds to the basal level of keratinocytic cytosoluble factors present under our culturing conditions.

The presence of sodium butyrate inhibits this “basal” production of MMP-1 by a factor of 3.

In the UV-B condition, an overproduction of MMP-1 is obtained, by a factor of 3 relative to the control condition (non-irradiated), due to the presence of photoinduced keratinocytic cytosoluble factors.

Sodium butyrate, added to the conditioned medium of irradiated keratinocytes, is capable of preventing this stimulatory effect on MMP-1 production, by inhibiting the production of photoinduced keratinocytic cytosoluble factors present in the medium.

These results thus show that sodium butyrate is an “indirect” inhibitor of choice for cosmetic applications directed towards preventing and/or treating collagen degradation in the dermis associated with exposure to sunlight.

### Example 3

### Formulations

Composition 1: Facial care cream
The following oil-in-water emulsion is prepared in a conventional
manner (% by weight):

| | | |
|---|---|---|
| Sodium butyrate | | 1% |
| Lycopene (in the form of Lycomato ® | | 0.001% |
| containing 10% lycopene in a tomato | | |
| oleoresin, sold by Lycored ® | | |
| Glyceryl stearate | | 2% |
| Polysorbate 60 (Tween 60 ® sold by ICI) | | 1% |
| Stearic acid | | 1.4% |
| Triethanolamine | | 0.7% |
| Carbomer | | 0.4% |
| Liquid fraction of shea butter | | 12% |
| Perhydrosqualene | | 12% |
| Antioxidant | qs | |
| Fragrance | qs | |
| Preservative | qs | |
| Water | qs | 100% |

Composition 2: Facial care gel

| | | |
|---|---|---|
| Sodium butyrate | | 10⁻⁵% |
| Hydroxypropylcellulose (Klucel H ® sold | | 1.00% |
| by Hercules) | | |
| Antioxidant | | 0.05% |
| Isopropanol | | 40.00% |
| Preservative | | 0.30% |
| Water | qs | 100.00% |

Composition 3: Lotion for preventing hair loss

| | | |
|---|---|---|
| Monoacetone glucose-3-butyrate | | 1% |
| Lycopene (in the form of Lycomato ® | | 0.0001% |
| containing 10% lycopene in a tomato | | |
| oleoresin, sold by Lycored ®) | | |
| Propylene glycol | | 30% |
| Ethanol | | 55% |
| Water | qs | 100% |

Composition 4: Plant or animal gelatin gel capsule

| | mg/gel capsule |
|---|---|
| Sodium butyrate | 10 |
| Starch | 198 |
| Magnesium stearate | 2.5 |

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A regime or regimen for preventing and/or combating the degradation of collagen fibers induced by solar radiation characterized by a UV-A/UV-B ratio ranging from 10 and 17, comprising administering to an individual in need of such treatment, a thus effective amount of at least one inhibitor of the production of photoinduced keratinocytic cytosoluble factors.

2. The regime or regimen as defined by claim 1, said at least one inhibitor of the production of photoinduced keratinocytic cytosoluble factors having been selected according to the process comprising the following steps:
(a) normal human keratinocytes cultured as a monolayer are subjected to a simulated solar radiation or to a UV radiation, characterized by a UV-A/UV-B ratio ranging from 10 to 17;
(b) the irradiated or non-irradiated keratinocytes are placed in contact with a culture medium containing or not containing the test product;
(c) placing the conditioned medium obtained in (b) in contact with normal human fibroblasts cultured as a monolayer;
(d) measuring the production of interstitial collagenase MMP-1 in the fibroblast culture supernatant obtained in (c), and comparing same with a control comprising a culture of normal human fibroblasts placed in contact with a conditioned medium of non-irradiated keratinocytes; and
(e) selecting the products for which the measurement of the production of interstitial collagenase MMP-1 is at least 10% less than that obtained for the control.

3. The regime or regimen as defined by claim 1, said at least one inhibitor of the production of photoinduced keratinocytic cytosoluble factors comprising an inflammatory cytokine production inhibitor, a cyclooxygenase inhibitor, a histone deacetylase inhibitor, or mixture thereof.

4. A regime or regimen for preventing and/or combating the photoinduced degradation of collagen fibers, comprising administering to an individual in need of such treatment, a thus effective amount of at least one inhibitor of photoinduced keratinocytic cytosoluble factors, selected from the group consisting of a histone deacetylase inhibitor, a cyclooxygenase inhibitor, and mixtures thereof.

5. The regime or regimen as defined by claim 3, comprising administering a histone deacetylase inhibitor selected from the group consisting of α-lipoic acid, trichostatin A, a C2 to C6 short-chain fatty acid, propionic acid and/or derivative and/or salt thereof, butyric acid and/or derivative and/or salt thereof, and mixtures thereof.

6. The regime or regimen as defined by claim 5, comprising administering sodium butyrate.

7. The regime or regimen as defined by claim 1, the effective amount of at least one inhibitor of the production of photoinduced keratinocytic cytosoluble factors reducing or inhibiting the production of interstitial collagenase MMP-1 in the dermis, via a paracrine mechanism.

8. The regime or regimen as defined by claim 1, comprising coadministering at least one direct inhibitor of MMP-1 production.

9. The regime or regimen as defined by claim 1, for preventing and/or combating collagen fiber degradation induced in the presence of a sub-erythemal or erythemal natural sunlight exposure.

10. The regime or regimen as defined by claim 1, to prevent thinning of the skin, and loss of tonicity and/or loss of elasticity of the skin.

11. The regime or regimen as defined by claim 1, comprising orally administering or topically applying said at least one inhibitor.

12. A topically applicable cosmetic/dermatological composition for preventing and/or combating the degradation of collagen fibers induced by solar radiation and formulated as an aqueous-alcoholic or oily solution, an oil-in-water or water-in-oil or multiple emulsion, an aqueous or anhydrous gel, microcapsules or microparticles, or a vesicular dispersion of ionic and/or nonionic type, comprising a thus effective amount of at least one histone deacetylase inhibitor selected from the group consisting of butyric acid and the salts, derivatives and mixtures thereof, in a topically applicable, cosmetically/dermatologically acceptable medium therefor.

13. The cosmetic/dermatological composition as defined by claim 12, said at least one histone deacetylase inhibitor comprising sodium butyrate.

14. A cosmetic/dermatological composition for preventing and/or combating the degradation of collagen fibers induced by solar radiation, comprising a thus effective amount of (i) at least one inhibitor of the production of photoinduced keratinocytic cytosoluble factors and (ii) at least one direct inhibitor of MMP-1 production, formulated into (iii) a physiologically acceptable medium therefor.

15. The cosmetic/dermatological composition as defined by claim 14, said at least one direct inhibitor of MMP-1 production being selected from the group consisting of TIMP-1; lycopene or an isoflavone and derivatives thereof; retinol or its derivatives, retinoic acid and its derivatives; extracts of sage, blueberry or rosemary; adapalene or similar peptides and/or Batimastat derivatives ((BB 94)=[4-(N-hydroxyamino)-2R-isobutyl-3S-(thiophen-2-ylthiomethyl)succinyl]-L-phenylalanine-N-methylamide), Marimastat ((BB 2516)=[2S-[N4(R*),2R*,3S]]-N4[2,2-dimethyl-1-[(methylamino)carbonyl]propyl]-N 1,2-dihydroxy-3-(2-methylpropyl)butanediamide); sapogenins, diosgenin, hecogenin, smilagenin, sarsapogenin, tigogenin, yamogenin and yuccagenin; genistein and quercetin; tetracyclines and derivatives thereof, minocycline, roliteracycline, chlortetracycline, methacycline, oxytetracycline, doxycycline, demeclocycline and corresponding salts; oligopeptides and lipopeptides, and lipoamino acids.

16. The cosmetic/dermatological composition as defined by claim 14, comprising from 10⁻¹²% to 5% by weight of said at least one inhibitor of the production of photoinduced keratinocytic cytosoluble factors.

17. The cosmetic/dermatological composition as defined by claim 14, comprising from 10⁻¹⁰% to 2% by weight of said at least one inhibitor of the production of photoinduced keratinocytic cytosoluble factors.

18. The cosmetic/dermatological composition as defined by claims 12 or 14, further comprising an active agent selected from the group consisting of an agent that stimulates the synthesis of dermal macromolecules or that prevents their degradation, an agent that stimulates collagen synthesis or prevents its degradation, an agent that promotes hair growth and/or limits hair loss, an antioxidant, a UV-screening agent, and mixtures thereof.

19. The cosmetic/dermatological composition as defined by claim 14, formulated for oral administration.

20. The cosmetic/dermatological composition as defined by claim 14, formulated for topical application onto the skin and/or scalp.

21. A regime or regimen for inducing and/or stimulating hair growth and/or for reducing hair loss, comprising administering or topically applying onto the scalp of an individual in need of such treatment, a thus effective amount of at least one inhibitor of the production of photoinduced keratinocytic cytosoluble factors.

22. The regime or regimen as defined by claim 1, said at least one inhibitor of the production of photoinduced keratinocytic cytosoluble factors having been selected according to the process comprising the following steps:
(a) normal human keratinocytes cultured as a monolayer are subjected to a simulated solar radiation or to a UV radiation, characterized by a UV-A/UV-B ratio ranging from 10 to 17;
(b) the irradiated or non-irradiated keratinocytes are placed in contact with a culture medium containing or not containing the test product;
(c) placing the conditioned medium obtained in (b) in contact with normal human fibroblasts cultured as a monolayer;
(d) measuring the production of interstitial collagenase MMP-1 in the fibroblast culture supernatant obtained in (c), and comparing same with a control comprising a culture of normal human fibroblasts placed in contact with a conditioned medium of non-irradiated keratinocytes; and
(e) selecting the products for which the measurement of the production of interstitial collagenase MMP-1 is at least 30% less than that obtained for the control.
